(19) United States
(12) Patent Application Publication
(10) Pub. No.: US 2016/0045360 A1
(43) Pub. Date: Feb. 18, 2016

(54) Athletic Garment Assembly

(71) Applicant: James White, Stockton, CA (US)

(72) Inventor: James White, Stockton, CA (US)

(21) Appl. No.: 14/457,716

(22) Filed: Aug. 12, 2014

(51) Int. Cl. A61F 5/44; A61F 5/455; A61F 5/453; A41B 9/00; A41B 9/02

(52) U.S. Cl. CPC A61F 5/4408; A41B 9/004; A41B 9/02; A61F 5/453; A61F 5/4556

(57) ABSTRACT

An athletic garment assembly for collecting urine during athletic activity includes a jock strap that may be worn by a user. The jock strap is structured so a crotch of the jock strap covers the user's genitalia when the user wears the jock strap. A funnel is coupled to the jock strap. The funnel engages the user's genitalia so the funnel may receive urine from the user while the user engages in athletic activity. A bladder is coupled to the funnel so the bladder may contain the urine.

### BACKGROUND OF THE DISCLOSURE

### Field of the Disclosure

The disclosure relates to athletic devices and more particularly pertains to a new athletic device for collecting urine during athletic activity.

### SUMMARY OF THE DISCLOSURE

An embodiment of the disclosure meets the needs presented above by generally comprising a jock strap that may be worn by a user. The jock strap is structured so a crotch of the jock strap covers the user's genitalia when the user wears the jock strap. A funnel is coupled to the jock strap. The funnel engages the user's genitalia so the funnel may receive urine from the user while the user engages in athletic activity. A bladder is coupled to the funnel so the bladder may contain the urine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is an inside perspective view of a athletic garment assembly according to an embodiment of the disclosure.

FIG. 2 is a perspective view of an embodiment of the disclosure.

FIG. 3 is a front view of an embodiment of the disclosure.

FIG. 4 is an exploded front view of an embodiment of the disclosure.

FIG. 5 is a front perspective view of an embodiment of the disclosure.

### DESCRIPTION OF THE PREFERRED EMBODIMENT

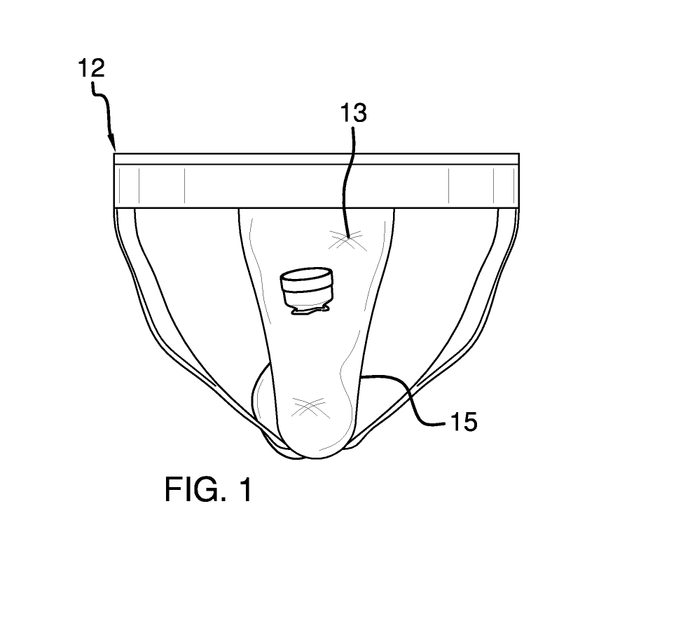

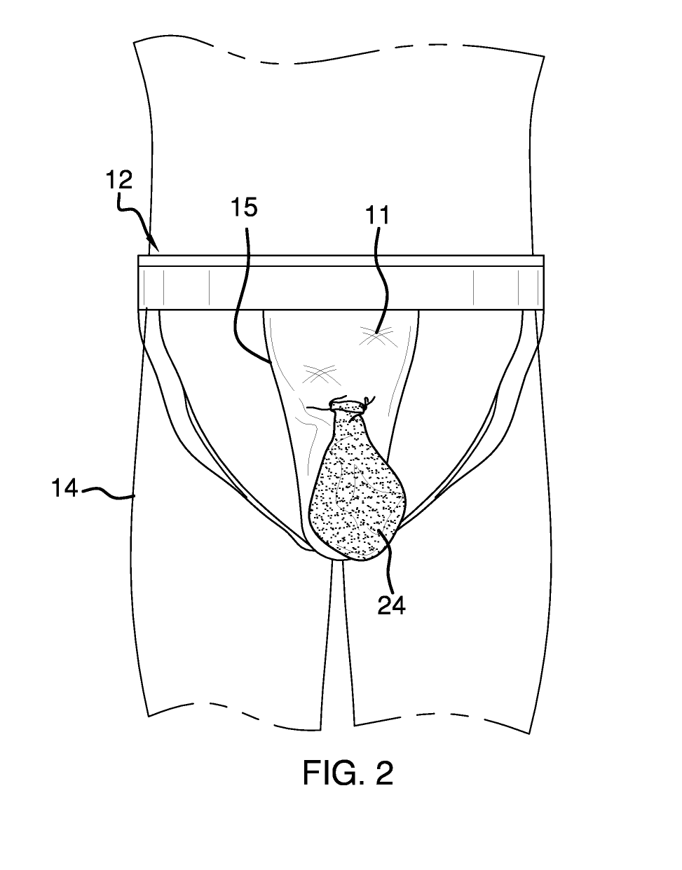

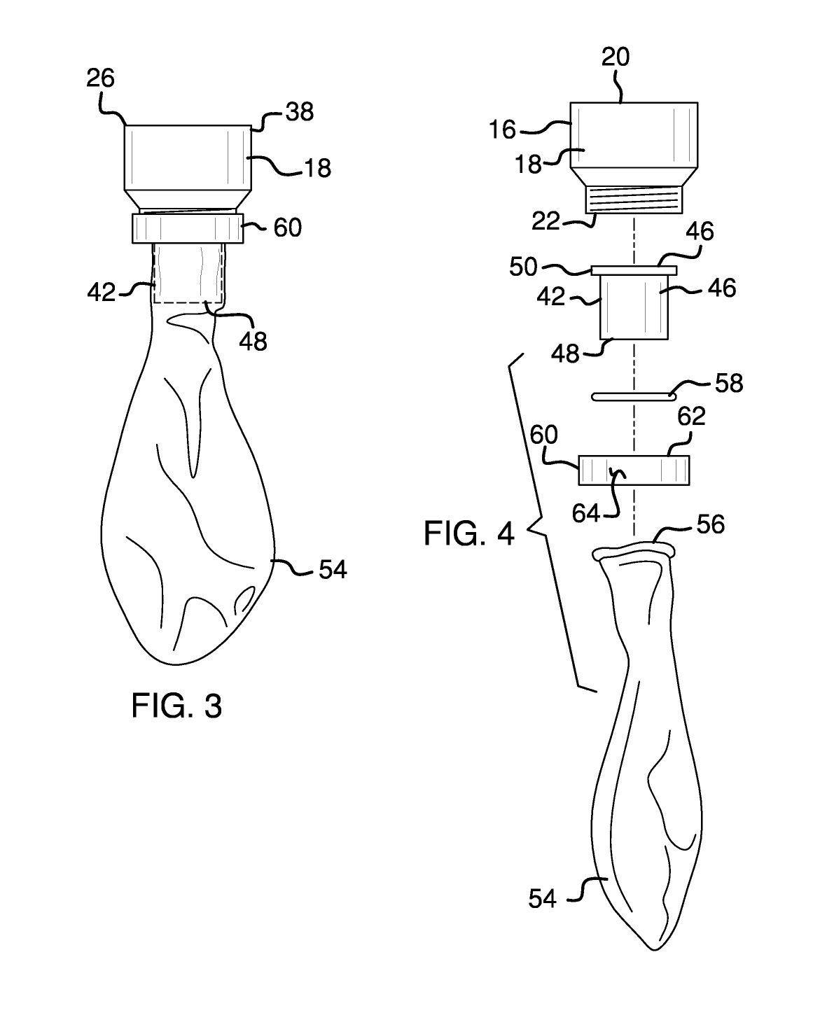

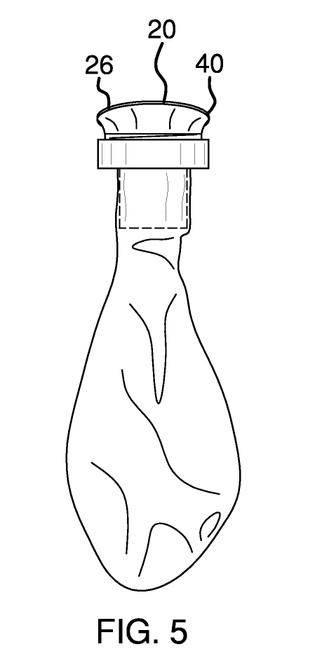

With reference now to the drawings, and in particular to FIGS. 1 through 5 thereof, a new athletic device embodying the principles and concepts of an embodiment of the disclosure and generally designated by the reference numeral 10 will be described.

As best illustrated in FIGS. 1 through 5, the athletic garment assembly 10 generally comprises a jock strap 12 that may be worn by a user 14. The jock strap 12 is structured so a crotch 15 of the jock strap 12 covers the user's genitalia when the user 14 wears the jock strap 12. The jock strap 12 may be an athletic support of any conventional design.

A funnel 16 is provided. The funnel 16 has an outer wall 18 extending between each of a first end 20 and a second end 22 of the funnel 16. Each of the first 20 and second 22 ends of the funnel 16 are open. The funnel 16 is substantially hollow. The funnel 16 is extendable through a front side 11 and a back side 13 of the crotch 15. The second end 22 of the funnel 16 has a diameter that is less than a diameter of the first end 20 of the funnel 16.

The first end 20 of the funnel 16 engages the user's genitalia and the second end 22 of the funnel 16 is exposed on the crotch 15 when the user 14 wears the jock strap 12. Moreover, the funnel 16 may receive urine 24 from the user 14 while the user 14 engages in athletic activity. The funnel 16 is one of a pair of funnels 26. The first end 20 of a first one 38 of the funnels 26 is shaped to engage male genitalia. The first end 20 of a second one 40 of the funnels 26 is shaped to engage female genitalia.

A coupler 42 is provided. The coupler 42 has an exterior wall 44 extending between a top end 46 and a bottom end 48 of the coupler 42. Each of the top 46 and bottom 48 ends of the coupler 42 is open. The coupler 42 is substantially hollow.

A lip 50 extends outwardly from the exterior wall 44 of the coupler 42. The lip 50 is coextensive with the top end 46 of the coupler 42. The top end 46 of the coupler 42 engages the second end 22 of the funnel 16. The coupler 42 is fluidly coupled to the funnel 16.

A bladder 54 has a top end 56. The top end 56 of the bladder 54 is open. Additionally, the top end 56 of the bladder 54 insertably receives the bottom end 48 of the coupler 42. The bladder 54 is fluidly coupled to the funnel 16 so the bladder 54 may contain the urine 24.

A gasket 58 is provided. The gasket 58 has a ring shape. The gasket 58 slides over the top end 56 of the bladder 54 on the coupler 42 and engages the lip 50 on the coupler 42. The gasket 58 may be comprised of a resiliently compressible material.

A retainer 60 is provided. An inner surface 62 of the retainer 60 is curvilinear with respect to an outer surface 64 of the retainer 60. The retainer 60 forms a closed loop. The retainer 60 threadably engages the second end 22 of the funnel 16 after the bladder 54 is positioned on the coupler 42. The coupler 42 and the bladder 54 are each removably retained on the funnel 16. Additionally, the gasket 58 is compressed between the retainer 60 and the lip 50 on the coupler 42. The gasket 58 forms a fluid impermeable seal between the funnel 16 and the bladder 54.

In use, the user 14 wears the jock strap 12 when the user 14 plans to engage in athletic activities that restrict the user's 14 access to a bathroom. The user 14 urinates into the first end 20 of the funnel 16 while the user 14 participates in the athletic activity. The bladder 54 is removed from the funnel 16 and emptied after the user 14 completes the athletic activity.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An athletic garment assembly comprising:
a jock strap configured to be worn by a user, said jock strap being structured such that a crotch of said jock strap covers the user's genitalia when the user wears said jock strap;
a funnel coupled to said jock strap, said funnel engaging the user's genitalia such that said funnel is configured to receive urine from the user while the user engages in athletic activity; and
a bladder coupled to said funnel such that said bladder is configured to contain the urine.

2. The athletic garment assembly according to claim 1, further comprising said funnel having an outer wall extending between each of a first end and a second end of said funnel, each of said first and second ends of said funnel being open, said funnel being substantially hollow.

3. The athletic garment assembly according to claim 2, further comprising said funnel extending through said crotch such that said first end of said funnel engages the user's genitalia and said second end of said funnel is exposed on said crotch when the user wears said jock strap.

4. The athletic garment assembly according to claim 3, further comprising a coupler having an exterior wall extending between a top end and a bottom end of said coupler, each of said top and bottom ends of said coupler being open, said coupler being substantially hollow.

5. The athletic garment assembly according to claim 4, further comprising said top end of said coupler engaging said second end of said funnel such that said coupler is fluidly coupled to said funnel.

6. The athletic garment assembly according to claim 1, further comprising said bladder having a top end, said top end of said bladder being open.

7. The athletic garment assembly according to claim 6, further comprising said top end of said bladder insertably receiving a bottom end of a coupler such that said bladder is fluidly coupled to said funnel.

8. The assembly according to claim 7, further comprising a retainer, an inner surface of said retainer being curvilinear with respect to an outer surface of said retainer such that said retainer forms a closed loop.

9. The athletic garment assembly according to claim 8, further comprising said retainer threadably engaging a second end of said funnel after said bladder is positioned on said coupler such that said coupler and said bladder are each removably retained on said funnel.

10. An athletic garment assembly comprising:
a jock strap configured to be worn by a user, said jock strap being structured such that a crotch of said jock strap covers the user's genitalia when the user wears said jock strap;
a funnel, said funnel having an outer wall extending between each of a first end and a second end of said funnel, each of said first and second ends of said funnel being open, said funnel being substantially hollow, said funnel extending through said crotch such that said first end of said funnel engages the user's genitalia and said second end of said funnel is exposed on said crotch when the user wears said jock strap wherein said funnel is configured to receive urine from the user while the user engages in athletic activity;
a coupler having an exterior wall extending between a top end and a bottom end of said coupler, each of said top and bottom ends of said coupler being open, said coupler being substantially hollow, said top end of said coupler engaging said second end of said funnel such that said coupler is fluidly coupled to said funnel;
a bladder having a top end, said top end of said bladder being open, said top end of said bladder insertably receiving said bottom end of said coupler such that said bladder is fluidly coupled to said funnel wherein said bladder is configured to contain the urine; and
a retainer, an inner surface of said retainer being curvilinear with respect to an outer surface of said retainer such that said retainer forms a closed loop, said retainer threadably engaging said second end of said funnel after said bladder is positioned on said coupler such that said coupler and said bladder are each removably retained on said funnel.
